(19) United States
(12) Patent Application Publication
(10) Pub. No.: US 2005/0272748 A1
(43) Pub. Date: Dec. 8, 2005

(54) 2-Mercapto-substituted triazolopyrimidines, methods for the production thereof, the use of the same for controlling patogenic fungi, and agents containing said compounds

(75) Inventors: Jordi Tormo I. Blasco, Laudenbach (DE); Carsten Blettner, Mannheim (DE); Bernd Muller, Frankenthal (DE); Markus Gewehr, Kastellaun (DE); Wassilios Grammenos, Ludwigshafen (DE); Thomas Grote, Wachenheim (DE); Andreas Gypser, Mannheim (DE); Joachim Rheinheimer, Ludwigshafen (DE); Peter Schafer, Ottersheim (DE); Frank Schieweck, Hessheim (DE); Anja Schwogler, Mannheim (DE); Eberhard Ammermann, Heppenheim (DE); Siegfried Strathmann, Limburgerhoff (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF Aktiengesellschaft, 67056 Ludwigshafen (DE)

(21) Appl. No.: 10/531,980

(22) PCT Filed: Nov. 14, 2003
(86) PCT No.: PCT/EP03/12773

§ 371 Date: Apr. 20, 2005

(30) Foreign Application Priority Data
Nov. 15, 2002 (DE) 102 53 593.0
Jan. 31, 2003 (DE) 103 04 076.5

(51) Int. Cl. A61K 31/519; C07D 487/04

(52) U.S. Cl. 514/259.31; 544/263

(57) ABSTRACT

2-Mercapto-substituted triazolopyrimidines of the formula I 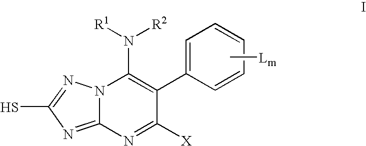 in which the substituents are as defined below: L is halogen, cyano, nitro, alkyl, alkenyl, alkynyl, haloalkyl, haloalkenyl, alkoxy, alkenyloxy, alkynyloxy, haloalkoxy or —C(═O)-A; A is hydrogen, hydroxyl, alkyl, alkenyl, alkoxy, haloalkoxy, alkylamino or dialkylamino; m is 0, 1, 2, 3, 4 or 5; X is halogen, cyano, alkyl, haloalkyl, alkoxy or haloalkoxy;
R¹, R² are hydrogen, alkyl, haloalkyl, cycloalkyl, halocycloalkyl, alkenyl, alkadienyl, haloalkenyl, cycloalkenyl, alkynyl, haloalkynyl or cycloalkynyl, phenyl, naphthyl or a five- to ten-membered saturated, partially unsaturated or aromatic heterocycle which contains one to four hetero atoms from the group consisting of O, N and S; R¹ and R² together with the nitrogen atom to which they are attached may also form a five- or six-membered ring which may be interrupted by an atom from the group consisting of O, N and S;
   where R¹ and/or R² may be substituted as stated in the description; processes for preparing these compounds, compositions comprising them and their use for controlling phytopathogenic harmful fungi are described.

The present invention relates to 2-mercapto-substituted triazolopyrimidines of the formula I 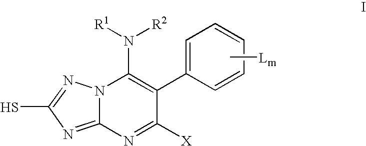
in which the substituents are as defined below:
L independently of one another are halogen, cyano, nitro, C₁-C₆-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy or —C(═O)-A;
   A is hydrogen, hydroxyl, C₁-C₈-alkyl, C₂-C₈-alkenyl, C₁-C₈-alkoxy, C₁-C₆-haloalkoxy, C₁-C₈-alkylamino or di-(C₁-C₈-alkyl)amino;
m is 0, 1, 2, 3, 4 or 5;
X is halogen, cyano, C₁-C₄-alkyl, C₁-C₄-haloalkyl, C₁-C₄-alkoxy or C₁-C₂-haloalkoxy;
R¹, R² independently of one another are hydrogen, C₁-C₈-alkyl, C₁-C₈-haloalkyl, C₃-C₆-cycloalkyl, C₃-C₆-halocycloalkyl, C₂-C₈-alkenyl, C₄-C₁₀-alkadienyl, C₂-C₈-haloalkenyl, C₃-C₆-cycloalkenyl, C₂-C₈-alkynyl, C₂-C₈-haloalkynyl or C₃-C₆-cycloalkynyl, phenyl, naphthyl or a five- to ten-membered saturated, partially unsaturated or aromatic heterocycle which contains one to four hetero atoms from the group consisting of O, N and S,
   R¹ and R² together with the nitrogen atom to which they are attached may also form a five- or six-membered ring which may be interrupted by one atom from the group consisting of O, N and S and/or may carry one or more substituents from the group consisting of halogen, C₁-C₆-alkyl, C₁-C₆-haloalkyl and oxy-C₁-C₃-alkyleneoxy or in which a nitrogen atom and an adjacent carbon atom may be linked by a C₁-C₄-alkylene chain;
   where R¹ and/or R² may be substituted by one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₃-C₆-cycloalkyl, phenyl, naphthyl, a five- to ten-membered saturated, partially unsaturated or aromatic heterocycle which contains one to four hetero atoms from the group consisting of O, N and S,
   where these aliphatic, alicyclic or aromatic groups for their part may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups in these radicals contain 2 to 8 carbon atoms;
   and/or one to three of the following radicals:
   cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the alkyl radicals preferably contain 6 to 10 ring members and the hetaryl radicals 5 or 6 ring members, where the cyclic systems may be partially or fully halogenated or substituted by alkyl or haloalkyl groups,
      and their salts.

Moreover, the invention relates to processes for preparing these compounds, to compositions comprising them and to their use for controlling phytopathogenic harmful fungi.

6-Phenyl-7-aminotriazolopyrimidines are commonly-known from EP-A 71 792 and EP-A 550 113. WO 02/088127 discloses 2-thiotriazolopyrimidines. The compounds described in the publications mentioned are known to be suitable for controlling harmful fungi.

However, in many cases their activity is unsatisfactory.

It is an object of the present invention to provide compounds having improved activity and/or a broader activity spectrum.

We have found that this object is achieved by the compounds defined at the outset. Furthermore, we have found processes and intermediates for preparing these compounds, compositions comprising them and methods for controlling harmful fungi using the compounds I.

The compounds of the formula I differ from those of the publications mentioned above by the mercapto group in the 2-position of the triazolopyrimidine skeleton.

The compounds of the formula I have a higher activity against harmful fungi than the known compounds.

The compounds according to the invention can be obtained by different routes. Advantageously, they are obtained by reacting sulfoxides of the formula II with trifluoroacetic anhydride (TFA) under the conditions known from J. Fluorine Chem. (1996), 159 and J. Het. Chem. (1988), 1007.

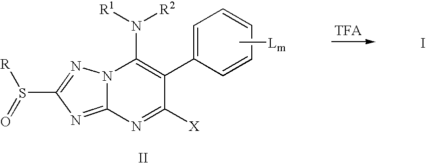

In formula II, the variables are as defined for formula I, the form of group R being of lesser importance; for practical reasons preference is given to a C₁-C₄-alkyl group, in particular methyl, or a benzyl group which is unsubstituted or substituted by one or more groups R^{b}.

Sulfones of the formula III provide alternative access route to the compounds of the formula I. By substitution of the sulfone group with S²⁻ or SH⁻ nucleophiles under the conditions known from J. Het. Chem. (1990), 839 and Chem. Pharm. Bull. (1976), 136, the compounds of the formula I are obtained. The definitions of the variables in formula III correspond to those in formula II.

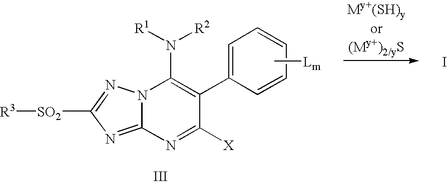

Use is usually made of thiolates (M^{y+})_{y}SH or sulfides (M^{y+})_{2/y}S where M is a cation from the group of the alkali metals or alkaline earth metals of valency y or an ammonium group NR₄⁺ (R═H or C₁-C₄-alkyl). In this process, it is particularly advantageous to employ NaSH×H₂O, Na₂S or (NH₄)₂S, in particular NaSH×H₂O.

Compounds of the formula I can also be obtained from thio compounds of the formula IV.

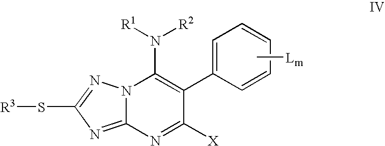

In formula IV, the definitions of the variables correspond to those for formula I, whereas group R³ is a removable protective group. For practical reasons, preference is given to a C₁-C₆-alkyl group or a benzyl group which is unsubstituted or substituted by one or more groups R^{b} [cf. Greene, Protective Groups in Organic Chemistry, J. Wiley & Sons, pp. 195-217 (1981); J. Org. Chem., 43 (1978), 1197; Tetrahedron (2001), 1897]. Particular preference is given to compounds of the formula IV in which R³ is benzyl [IV.1], p-acetoxybenzyl [IV.2] or p-methoxybenzyl [IV.3].

The conversion of the thio compounds of the formula IV, in particular of the formula IV.1, into compounds of the formula I is carried out, for example, by reduction with alkali metals, in particular with sodium, in general in the presence of a base. A suitable base is, in particular, liquid ammonia, which also serves as solvent. Other suitable solvents are ethers, such as tetrahydrofuran, or alcohols, such as ethanol or butanol, or mixtures thereof (cf.: J. Chem. Soc., Perkin Trans. I (1977), 1421; J. Org. Chem. (1991), 6672; DE-A 35 45 124].

An alternative access route to compounds of the formula I from the thio compounds of the formula IV, in particular of the formula IV.1, consists in reacting IV with Lewis acids such as AlCl₃ in an inert solvent under the conditions known from J. Chem. Soc., Perkin Trans. I (1980), 1029 and JP 5-830 316.

The protective group in IV, in particular IV.1, can also be removed using HF in solvents such as, for example, anisole [cf.: Bull. Chem. Soc. Jpn. (1967), 2164].

The protective group in IV, in particular in IV.2, is advantageously removed under basic conditions using, in particular, alkali metal hydroxides or alkaline earth metal hydroxides, such as, for example, NaOH [cf.: J. Org. Chem. (1978), 1197].

The protective group in IV, in particular IV.3, is advantageously removed using the 2-chlorosulfenyl-3-nitropyridine/Bu₃P system [cf.: Tetrahedron (2001), 1897].

The starting materials, required for preparing the compounds I, of the formulae II, III and IV in which X is halogen, are known from the literature [cf. WO 02/088127] or can be prepared in accordance with the literature cited.

Compounds of the formula I in which X is halogen, in particular chlorine (formula I.A) are a preferred subject matter of the invention.

Compounds I in which X is cyano, C₁-C₄-alkoxy or C₁-C₂-haloalkoxy (formula I.B) can be prepared in an advantageous manner from starting materials of the formula IV in which X is halogen (formula IV.A) via the compounds IV.B, by the routes illustrated below.

Compounds of the formula I in which X is cyano, C₁-C₆-alkoxy or C₁-C₂-haloalkoxy (formula I.B) can be prepared in an advantageous manner from compounds IV in which X is halogen [Hal], preferably chlorine, which compounds correspond to the formula IV.A.

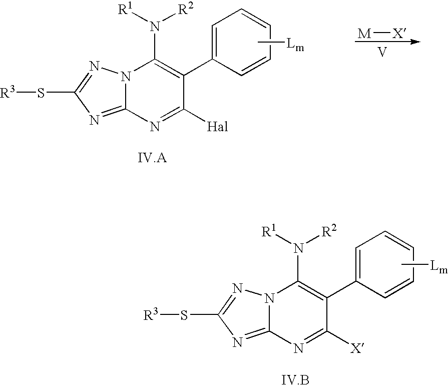

Compounds IV.A are reacted with compounds M-X′ (formula V) to give compounds IV.B. Depending on the meaning of the group X′ to be introduced, the compounds V are inorganic cyanides or alkoxides. The reaction is advantageously carried out in the presence of an inert solvent. In the formula V, the cation M is of minor importance; for practical reasons, preference is usually given to ammonium, tetraalkylammonium or alkali metal or alkaline earth metal salts.

The reaction temperature is usually from 0 to 120° C., preferably from 10 to 40° C. [cf. J. Heterocycl. Chem. 12 (1975), 861-863].

Suitable solvents include ethers, such as dioxane, diethyl ether and, preferably, tetrahydrofuran, halogenated hydrocarbons, such as dichloromethane, and aromatic hydrocarbons, such as toluene.

Conversion of the compounds IV.B into compounds I.B is carried out under the reaction conditions described further above for the compounds IV (in particular IV.1 to IV.3).

Compounds I in which X is C₁-C₄-alkyl or C₁-C₄-haloalkyl (formula I.C) can be prepared in an advantageous manner from starting materials of the formula IV.A via the compounds IV.C, by the routes illustrated below.

Compounds of the formula I.C in which X is C₁-C₄-alkyl can be obtained by coupling 5-halotriazolopyrimidines of the formula IV.A with organometallic reagents of the formula VI. In one embodiment of this process, the reaction is carried out under transition metal catalysis, such as Ni or Pd catalysis.

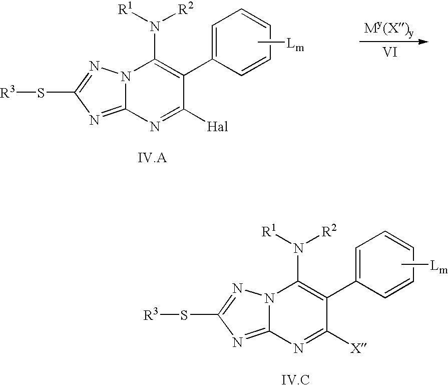

In formula VI, X″ is C₁-C₄-alkyl and M is a metal ion of the valency y, such as, for example, B, Zn or Sn. This reaction can be carried out, for example, analogous to the following methods: J. Chem. Soc., Perkin Trans. 1(1984), 1187, ibid. (1996), 2345; WO 99/41255; Aust. J. Chem. 43 (1990), 733; J. Org. Chem. 43 (1978), 358; J. Chem. Soc., Chem. Commun. (1979), 866; Tetrahedron Lett. 34 (1993), 8267; ibid. 33 (1992), 413.

Compounds of the formula I in which X is C₁-C₄-alkyl or C₁-C₄-haloalkyl (formula I.C) can advantageously also be obtained by the synthesis route below:

The 5-alkyl-7-hydroxy-6-phenyltriazolopyrimidines IX are obtained from aminotriazole derivatives VII and the keto ester VIII. In formula VIII, R is a C₁-C₄-alkyl group, in particular methyl or ethyl. Using the easily obtainable 2-phenylacetoacetic esters Villa where X″=CH₃, 5-methyl-7-hydroxy-6-phenyltriazolopyrimidines are obtained [cf. Chem. Pharm. Bull. 9 (1961), 801]. Some triazoles VII are commercially available or can be prepared under generally known conditions. The starting materials VIII are advantageously prepared under the conditions known from EP-A 10 02 788.

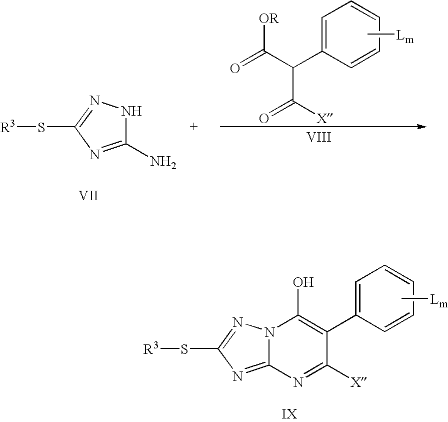

The 5-alkyl-7-hydroxy-6-phenyltriazolopyrimidines IX obtained in this manner are reacted with halogenating agents [HAL] to give 7-halotriazolopyrimidines of the formula X.

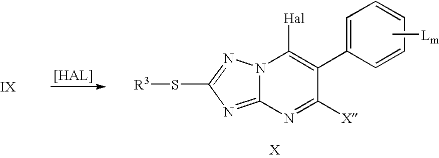

Preference is given to using chlorinating or brominating agents such as phosphorus oxybromide, phosphorus oxychloride, thionyl chloride, thionyl bromide or sulfuryl chloride. The reaction can be carried out in the absence or presence of a solvent. Customary reaction temperatures are from 0 to 150° C. or preferably from 80 to 125° C.

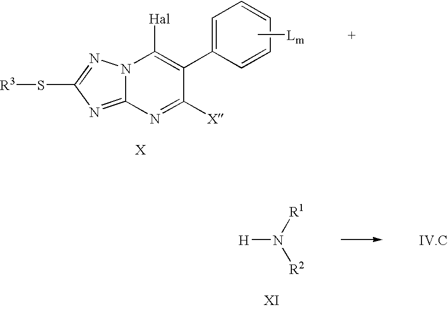

The reaction of X with amines XI is advantageously carried out at from 0° C. to 70° C., preferably from 10° C. to 35° C., with preference in the presence of an inert solvent such as an ether, for example dioxane, diethyl ether or, in particular, tetrahydrofuran, a halogenated hydrocarbon, such as dichloromethane, or an aromatic hydrocarbon, such as, for example, toluene [cf. WO 98/46608].

Preference is given to using a base, such as a tertiary amine, for example triethylamine, or an organic amine, such as potassium carbonate; it is also possible for excess amine of the formula XI to serve as base.

Alternatively, compounds of the formula IV.C can also be prepared from compounds IV.A and malonates of the formula XII. In formula XII, X′″ is hydrogen, C₁-C₃-alkyl or C₁-C₃-haloalkyl and R is C₁-C₄-alkyl. These compounds are converted into compounds of the formula XIII and decarboxylated to give compounds of the formula IV.C [cf. U.S. Pat. No. 5,994,360].

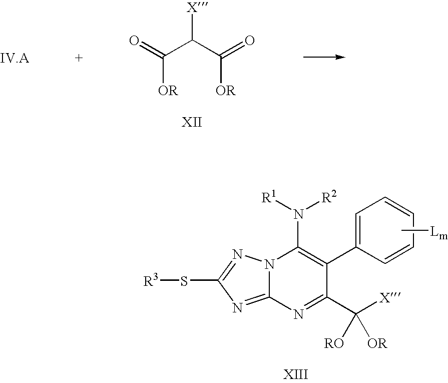

The malonates XII are known from the literature [J. Am. Chem. Soc. 64 (1942), 2714; J. Org. Chem. 39 (1974), 2172; Helv. Chim. Acta 61 (1978), 1565] or they can be prepared in accordance with the literature cited.

The subsequent hydrolysis of the ester XIII is carried out under generally customary conditions; depending on the different structural elements, alkaline or acidic hydrolysis of the compounds XIII may be advantageous. Partial or complete decarboxylation to IV.C may even take place under the conditions of the ester hydrolysis.

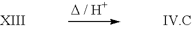

Usually, decarboxylation takes place at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tertbutyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isobutanol and tert-butanol, and also dimethyl sulfoxide, dimethylformamide and dimethylacetamide; with particular preference, the reaction is carried out in hydrochloric acid or acetic acid. It is also possible to use mixtures of the solvents mentioned.

The oxidation of the compounds IV.B or IV.C to give the sulfoxides of the formula II or the sulfones of the formula III in which X is cyano, C₁-C₄-alkoxy or C₁-C₂-haloalkoxy (formulae II.B or III.B) or C₁-C₄-alkyl or C₁-C₄-haloalkyl (formulae II.C or III.c) is usually carried out at temperatures of from −40° C. to 60° C., preferably from −40° C. to 40° C., in an inert organic solvent (cf. Synth. Commun. 16 (1986), 233ff.; WO 02/088127].

Suitable oxidizing agents are, for example, inorganic peroxides, such as hydrogen peroxide, or peroxocarboxylic acids, such as peracetic acid or perbenzoic acids, in particular meta-chloroperbenzoic acid.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plants, or in the harmful fungus to be controlled.

In the definitions of the symbols given in the formulae above, collective terms were used which are generally representative of the following substituents:
halogen: fluorine, chlorine, bromine and iodine;
alkyl: saturated straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 2, 4 or 6 carbon atoms (as mentioned above), where in these groups some or all of the hydrogen atoms may be replaced by halogen atoms as mentioned above; in partticular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals haying 2 to 4, 6, 8 or 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated straight-chain or branched hydrocarbon radicals having 2 to 10 carbon atoms and one or two double bonds in any position (as mentioned above), where in these groups some or all of the hydrogen atoms may be replaced by halogen atoms as mentioned above, in particular by fluorine, chlorine and bromine;
alkynyl: straight-chain or branched hydrocarbon groups having 2 to 4, 6, 8 or 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl; cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;
five- to ten-membered saturated, partially unsaturated or aromatic heterocycle which contains one to four heteroatoms from the group consisting of O, N and S:
   5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
   5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
   6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.
Alkylene: divalent unbranched chains of 3 to 5 CH₂ groups, for example CH₂, CH₂CH₂, CH₂CH₂CH₂, CH₂CH₂CH₂CH₂ and CH₂CH₂CH₂CH₂CH₂;
oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O.

The compounds of the formula I can also be present in the form of their agriculturally useful salts, the type of salt generally not being important. Suitable are, in general, the salts of those cations or the acid addition salts of those acids whose cations and anions, respectively, have no adverse effect on the fungicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-ylammonium, di-(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

The scope of the present invention includes the (R)- and (S)-isomers and the racemates of compounds of the formula I having chiral centers.

The particularly preferred embodiments of the intermediates with respect to the variables correspond to those of radicals Lₘ, R¹, R² and X of formula I.

With a view to the intended use of the triazolopyrimidines of the formula I, particular preference is given to the following meanings of the substituents, in each case on their own or in combination:

Preference is given to compounds I in which R¹ is C₁-C₄-alkyl, C₂-C₆-alkenyl or C₁-C₈-haloalkyl.

Preference is likewise given to compounds I in which R¹ is a 5- or 6-membered saturated or aromatic heterocycle which contains one or two hetero atoms from the group consisting of N, O and S and which may be substituted by one or two alkyl or haloalkyl groups.

Particular preference is given to compounds I in which R¹ is a group B 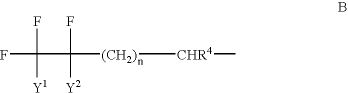
in which
Y¹ is hydrogen, fluorine or C₁-C₆-fluoroalkyl,
Y² is hydrogen or fluorine, or
Y¹ and Y² together form a double bond;
n is 0 or 1; and
R⁴ is hydrogen or methyl.

Moreover, preference is given to compounds I in which R¹ is C₃-C₆-cycloalkyl which may be substituted by C₁-C₄-alkyl.

Particular preference is given to compounds I in which R² is hydrogen.

Likewise, preference is given to compounds I in which R² is methyl or ethyl.

If R¹ and/or R² comprise haloalkyl or haloalkenyl groups having a center of chirality, the (S)-isomers are preferred for these groups. In the case of halogen-free alkyl or alkenyl groups having a center of chirality in R¹ or R², preference is given to the (R)-configured isomers.

Preference is furthermore given to compounds I in which R¹ and R² together with the nitrogen atom to which they are attached form a saturated or unsaturated five- or six-membered ring which may be interrupted by an atom from the group consisting of O, N and S and/or which may carry one or more substituents from the group consisting of halogen, C₁-C₆-alkyl, C₁-C₆-haloalkyl and oxy-C₁-C₃-alkyleneoxy or in which two adjacent ring members may be linked by a C₁-C₄-alkylene chain.

Particular preference is given to compounds I in which R¹ and R² together with the nitrogen atom to which they are attached form a piperidinyl, morpholinyl or thiomorpholinyl ring, in particular a piperidinyl ring which is unsubstituted or substituted by one to three halogen, C₁-C₄-alkyl or C₁-C₄-haloalkyl groups, in particular by 4-methyl.

Particular preference is furthermore given to compounds I in which R¹ and R² together with the nitrogen atom to which they are attached form a pyrrolidine ring which is unsubstituted or substituted by one or two halogen, C₁-C₄-alkyl or C₁-C₄-haloalkyl groups, in particular by 2-methyl.

Preference is given to compounds I in which at least one group L is located ortho to the point of attachment to the triazolopyrimidine skeleton; in particular to those compounds, in which n has the value 1, 2 or 3.

Preference is given to compounds I in which Lₙ is halogen, methyl, ethyl, C₁-haloalkyl, methoxy or —C(═O)-A, where A is hydrogen, hydroxyl, C₁-C₄-alkoxy, C₁-C₄-haloalkoxy, C₁-C₂-alkylamino or di-C₁-C₂-alkylamino.

Moreover, particular preference is given to compounds I in which the phenyl group substituted by Lₘ is the group A 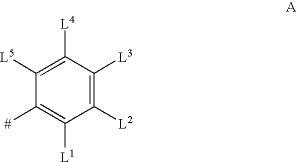
in which # is the point of attachment to the triazolopyrimidine skeleton and
L¹ is fluorine, chlorine, CH₃ or CF₃;
L², L⁴ independently of one another are hydrogen or fluorine;
L³ is hydrogen, fluorine, chlorine, cyano, CH₃ or COOCH₃; and
L⁵ is hydrogen, fluorine or CH₃.

Particular preference is given to compounds I in which X is halogen or C₁-C₄-alkyl, such as chlorine or methyl, in particular chlorine.

In particular with a view to their use, preference is given to the compounds I compiled in the tables below. Moreover, the groups mentioned for a substituent in the tables are, independently of the combination in which they are mentioned, a particularly preferred embodiment per se of the substituent in question.

Table 1

Compounds of the formula I in which X is chlorine, Lₘ is 2-fluoro-6-chloro and the combination of R¹ and R² corresponds for each compound to one row of table A

Table 2

Compounds of the formula I in which X is chlorine, Lₘ is 2,6-difluoro and the combination of R¹ and R² corresponds for each compound to one row of table A

Table 3

Compounds of the formula I in which X is chlorine, Lₘ is 2,6-dichloro and the combination of R¹ and R² corresponds for each compound to one row of table A

Table 4

Compounds of the formula I in which X is chlorine, Lₘ is 2-fluoro-6-methyl and the combination of R¹ and R² corresponds for each compound to one row of table A

Table 5

Compounds of the formula I in which X is chlorine, Lₘ is 2,4,6-trifluoro and the combination of R¹ and R² corresponds for each compound to one row of table A

Table 6

Compounds of the formula I in which X is chlorine, Lₘ is 2,6-difluoro-4-methoxy and the combination of R¹ and R² corresponds for each compound to one row of table A

Table 7

Compounds of the formula I in which X is chlorine, Lₘ is pentafluoro and the combination of R¹ and R² corresponds for each compound to one row of table A

Table 8

Compounds of the formula I in which X is chlorine, Lₘ is 2-methyl-4-fluoro and the combination of R¹ and R² corresponds for each compound to one row of table A

Table 9

Compounds of the formula I in which X is chlorine, Lₘ is 2-trifluoromethyl and the combination of R¹ and R² corresponds for each compound to one row of table A

Table 10

Compounds of the formula I in which X is chlorine, Lₘ is 2-methoxy-6-fluoro and the combination of R¹ and R² corresponds for each compound to one row of table A

Table 11

Compounds of the formula I in which X is chlorine, Lₘ is 2-chloro and the combination of R¹ and R² corresponds for each compound to one row of table A

Table 12

Compounds of the formula I in which X is chlorine, Lₘ is 2-fluoro and the combination of R¹ and R² corresponds for each compound to one row of table A

Table 13

Compounds of the formula I in which X is chlorine, Lₘ is 2,4-difluoro and the combination of R¹ and R² corresponds for each compound to one row of table A

Table 14

Compounds of the formula I in which X is chlorine, Lₘ is 2-fluoro-4-chloro and the combination of R¹ and R² corresponds for each compound to one row of table A

Table 15

Compounds of the formula I in which X is chlorine, Lₘ is 2-chloro-4-fluoro and the combination of R¹ and R² corresponds for each compound to one row of table A

Table 16

Compounds of the formula I in which X is chlorine, Lₘ is 2,3-difluoro and the combination of R¹ and R² corresponds for each compound to one row of table A

Table 17

Compounds of the formula I in which X is chlorine, Lₘ is 2,5-difluoro and the combination of R¹ and R² corresponds for each compound to one row of table A

Table 18

Compounds of the formula I in which X is chlorine, Lₘ is 2,3,4-trifluoro and the combination of R¹ and R² corresponds for each compound to one row of table A

Table 19

Compounds of the formula I in which X is chlorine, Lₘ is 2-methyl and the combination of R¹ and R² corresponds for each compound to one row of table A

Table 20

Compounds of the formula I in which X is chlorine, Lₘ is 2,4-dimethyl and the combination of R¹ and R² corresponds for each compound to one row of table A

Table 21

Compounds of the formula I in which X is chlorine, Lₘ is 2-methyl-4-chloro and the combination of R¹ and R² corresponds for each compound to one row of table A

Table 22

Compounds of the formula I in which X is chlorine, Lₘ is 2-fluoro-4-methyl and the combination of R¹ and R² corresponds for each compound to one row of table A

Table 23

Compounds of the formula I in which X is chlorine, Lₘ is 2,6-dimethyl and the combination of R¹ and R² corresponds for each compound to one row of table A

Table 24

Compounds of the formula I in which X is chlorine, Lₘ is 2,4,6-trimethyl and the combination of R¹ and R² corresponds for each compound to one row of table A

Table 25

Compounds of the formula I in which X is chlorine, Lₘ is 2,6-difluoro-4-cyano and the combination of R¹ and R² corresponds for each compound to one row of table A

Table 26

Compounds of the formula I in which X is chlorine, Lₘ is 2,6-difluoro-4-methyl and the combination of R¹ and R² corresponds for each compound to one row of table A

Table 27

Compounds of the formula I in which X is chlorine, Lₘ is 2,6-difluoro-4-methoxycarbonyl and the combination of R¹ and R² corresponds for each compound to one row of table A

Table 28

Compounds of the formula I in which X is chlorine, Lₘ is 2-trifluoromethyl-4-fluoro and the combination of R¹ and R² corresponds for each compound to one row of table A

Table 29

Compounds of the formula I in which X is chlorine, Lₘ is 2-trifluoromethyl-5-fluoro and the combination of R¹ and R² corresponds for each compound to one row of table A

Table 30

Compounds of the formula I in which X is chlorine, Lₘ is 2-trifluoromethyl-5-chloro and the combination of R¹ and R² corresponds for each compound to one row of table A

TABLE A

| No. | R¹ | R² |
|---|---|---|
| A-1 | H | H |
| A-2 | CH₂CH₃ | H |
| A-3 | CH₂CH₃ | CH₃ |
| A-4 | CH₂CH₃ | CH₂CH₃ |
| A-5 | CH₂CF₃ | H |
| A-6 | CH₂CF₃ | CH₃ |
| A-7 | CH₂CF₃ | CH₂CH₃ |
| A-8 | CH₂CCl₃ | H |
| A-9 | CH₂CCl₃ | CH₃ |
| A-10 | CH₂CCl₃ | CH₂CH₃ |
| A-11 | CH₂CH₂CH₃ | H |
| A-12 | CH₂CH₂CH₃ | CH₃ |
| A-13 | CH₂CH₂CH₃ | CH₂CH₃ |
| A-14 | CH₂CH₂CH₃ | CH₂CH₂CH₃ |
| A-15 | CH(CH₃)₂ | H |
| A-16 | CH(CH₃)₂ | CH₃ |
| A-17 | CH(CH₃)₂ | CH₂CH₃ |
| A-18 | (±) CH(CH₃)—CH₂CH₃ | H |
| A-19 | (±) CH(CH₃)—CH₂CH₃ | CH₃ |
| A-20 | (±) CH(CH₃)—CH₂CH₃ | CH₂CH₃ |
| A-21 | (S) CH(CH₃)—CH₂CH₃ | H |
| A-22 | (S) CH(CH₃)—CH₂CH₃ | CH₃ |
| A-23 | (S) CH(CH₃)—CH₂CH₃ | CH₂CH₃ |
| A-24 | (R) CH(CH₃)—CH₂CH₃ | H |
| A-25 | (R) CH(CH₃)—CH₂CH₃ | CH₃ |
| A-26 | (R) CH(CH₃)—CH₂CH₃ | CH₂CH₃ |
| A-27 | (±) CH(CH₃)—CH(CH₃)₂ | H |
| A-28 | (±) CH(CH₃)—CH(CH₃)₂ | CH₃ |
| A-29 | (±) CH(CH₃)—CH(CH₃)₂ | CH₂CH₃ |
| A-30 | (S) CH(CH₃)—CH(CH₃)₂ | H |
| A-31 | (S) CH(CH₃)—CH(CH₃)₂ | CH₃ |
| A-32 | (S) CH(CH₃)—CH(CH₃)₂ | CH₂CH₃ |
| A-33 | (R) CH(CH₃)—CH(CH₃)₂ | H |
| A-34 | (R) CH(CH₃)—CH(CH₃)₂ | CH₃ |
| A-35 | (R) CH(CH₃)—CH(CH₃)₂ | CH₂CH₃ |
| A-36 | (±) CH(CH₃)—C(CH₃)₃ | H |
| A-37 | (±) CH(CH₃)—C(CH₃)₃ | CH₃ |
| A-38 | (±) CH(CH₃)—C(CH₃)₃ | CH₂CH₃ |
| A-39 | (S) CH(CH₃)—C(CH₃)₃ | H |
| A-40 | (S) CH(CH₃)—C(CH₃)₃ | CH₃ |
| A-41 | (S) CH(CH₃)—C(CH₃)₃ | CH₂CH₃ |
| A-42 | (R) CH(CH₃)—C(CH₃)₃ | H |
| A-43 | (R) CH(CH₃)—C(CH₃)₃ | CH₃ |
| A-44 | (R) CH(CH₃)—C(CH₃)₃ | CH₂CH₃ |
| A-45 | (±) CH(CH₃)—CF₃ | H |
| A-46 | (±) CH(CH₃)—CF₃ | CH₃ |
| A-47 | (±) CH(CH₃)—CF₃ | CH₂CH₃ |
| A-48 | (S) CH(CH₃)—CF₃ | H |
| A-49 | (S) CH(CH₃)—CF₃ | CH₃ |
| A-50 | (S) CH(CH₃)—CF₃ | CH₂CH₃ |
| A-51 | (R) CH(CH₃)—CF₃ | H |
| A-52 | (R) CH(CH₃)—CF₃ | CH₃ |
| A-53 | (R) CH(CH₃)—CF₃ | CH₂CH₃ |
| A-54 | (±) CH(CH₃)—CCl₃ | H |
| A-55 | (±) CH(CH₃)—CCl₃ | CH₃ |
| A-56 | (±) CH(CH₃)—CCl₃ | CH₂CH₃ |
| A-57 | (S) CH(CH₃)—CCl₃ | H |
| A-58 | (S) CH(CH₃)—CCl₃ | CH₃ |
| A-59 | (S) CH(CH₃)—CCl₃ | CH₂CH₃ |
| A-60 | (R) CH(CH₃)—CCl₃ | H |
| A-61 | (R) CH(CH₃)—CCl₃ | CH₃ |
| A-62 | (R) CH(CH₃)—CCl₃ | CH₂CH₃ |
| A-63 | CH₂CF₂CF₃ | H |
| A-64 | CH₂CF₂CF₃ | CH₃ |
| A-65 | CH₂CF₂CF₃ | CH₂CH₃ |
| A-66 | CH₂(CF₂)₂CF₃ | H |
| A-67 | CH₂(CF₂)₂CF₃ | CH₃ |
| A-68 | CH₂(CF₂)₂CF₃ | CH₂CH₃ |
| A-69 | CH₂C(CH₃)═CH₂ | H |
| A-70 | CH₂C(CH₃)═CH₂ | CH₃ |
| A-71 | CH₂C(CH₃)═CH₂ | CH₂CH₃ |
| A-72 | CH₂CH═CH₂ | H |
| A-73 | CH₂CH═CH₂ | CH₃ |
| A-74 | CH₂CH═CH₂ | CH₂CH₃ |
| A-75 | CH(CH₃)CH═CH₂ | H |
| A-76 | CH(CH₃)CH═CH₂ | CH₃ |
| A-77 | CH(CH₃)CH═CH₂ | CH₂CH₃ |
| A-78 | CH(CH₃)C(CH₃)═CH₂ | H |
| A-79 | CH(CH₃)C(CH₃)═CH₂ | CH₃ |
| A-80 | CH(CH₃)C(CH₃)═CH₂ | CH₂CH₃ |
| A-81 | cyclopentyl | H |
| A-82 | cyclopentyl | CH₃ |
| A-83 | cyclopentyl | CH₂CH₃ |
| A-84 | cyclohexyl | H |
| A-85 | cyclohexyl | CH₃ |
| A-86 | cyclohexyl | CH₂CH₃ |

| | |
|---|---|
| A-87 | —(CH₂)₂CH═CHCH₂— |
| A-88 | —(CH₂)₂C(CH₃)═CHCH₂— |
| A-89 | —(CH₂)₂CH(CH₃)(CH₂)₂— |
| A-90 | —(CH₂)₂CHF(CH₂)₂— |
| A-91 | —(CH₂)₃CHFCH₂— |
| A-92 | —(CH₂)₂CH(CF₃)(CH₂)2— |
| A-93 | —(CH₂)₂O(CH₂)₂— |
| A-94 | —(CH₂)₂S(CH₂)₂— |
| A-95 | —(CH₂)₅— |
| A-96 | —(CH₂)₄— |
| A-97 | —CH₂CH═CHCH₂— |
| A-98 | —CH(CH₃)(CH₂)₃— |
| A-99 | —CH₂CH(CH₃)(CH₂)₂— |

The compounds I are suitable as fungicides. They are distinguished by an outstanding effectiveness against a broad spectrum of phytopathogenic fungi, especially from the classes of the Ascomycetes, Deuteromycetes, Phycomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Botrytis cinerea (gray mold) on strawberries, vegetables, ornamental plants and grapevines,
Cercospora arachidicola on peanuts,
Erysiphe cichoracearum and Sphaerotheca fuliginea on cucurbits,
Blumeria graminis (powdery mildew) on cereals,
Fusarium and Verticillium species on various plants,
Helminthosporium species on cereals,
Mycosphaerella species on bananas and peanuts, Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice, Rhizoctonia species on cotton, rice and lawns,
Septoria nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 10 g, preferably 0.01 to 2 g, per kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compounds. The active compounds are employed in a purity of from 90% to 100%, preferably 95% to 100% (according to NMR spectrum).

The following are examples of formulations:

1. Products for Dilution with Water
A Water-soluble concentrates (SL)
   10 parts by weight of a compound according to the invention are dissolved in water or in a water-soluble solvent. As an alternative, wetters or other auxiliaries are added. The active compound dissolves upon dilution with water.
B Dispersible concentrates (DC)
   20 parts by weight of a compound according to the invention are dissolved in cyclohexanone with addition of a dispersant, for example polyvinylpyrrolidone. Dilution with water gives a dispersion.
C Emulsifiable concentrates (EC)
   15 parts by weight of a compound according to the invention are dissolved in xylene with addition of calcium dodecylbenzenesulfonate and castor oil ethoxylate (in each case 5%). Dilution with water gives an emulsion.
D Emulsions (EW, EO)
   40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturax) and made into a homogeneous emulsion. Dilution with water gives an emulsion.
E Suspensions (SC, OD)
   In an agitated ball mill, 20 parts by weight of a compound according to the invention are comminuted with addition of dispersants, wetters and water or an organic solvent to give a fine active compound suspension. Dilution with water gives a stable suspension of the active compound.
F Water-dispersible granules and water-soluble granules (WG, SG) 50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.
G Water-dispersible powders and water-soluble powders (WP, SP) 75 parts by weight of a compound according to the invention are ground in a rotor-stator mill with addition of dispersants, wetters and silica gel. Dilution with water gives a stable dispersion or solution with the active compound.

2. Products to be Applied Undiluted
H Dustable powders (DP)
   5 parts by weight of a compound according to the invention are ground finely and mixed intimately with 95% of finely divided kaolin. This gives a dustable product.
I Granules (GR, FG, GG, MG)
   0.5 part by weight of a compound according to the invention is ground finely and associated with 95.5% carriers. Current methods are extrusion, spray-drying or the fluidized bed. This gives granules to be applied undiluted.
J ULV solutions (UL)
   10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, with which the compounds according to the invention can be used in conjunction, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthalisopropyl,
phenylpyrroles, such as fenpiclonil or fludioxonil,
sulfur,
other fungicides, such as acibenzolar-5-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below can be used to prepare further compounds I by appropriate modification of the starting compounds. The compounds thus obtained are listed in the tables below, together with physical data.

### Example 1

### Preparation of 3-benzylthio-5-amino-1,2,4-triazole

A solution of 0.17 mol of 5-amino-3-mercapto-1,2,4-triazole, 0.17 mol of benzyl bromide and 0.17 mol of NaOH in 100 ml of ethanol was stirred at 20-25° C. for about 4 hours. The solvent was distilled off and the residue was washed with water and dried, giving 31 g of the title compound of m.p. 107° C.

### Example 2

### Preparation of 5,7-dihydroxy-2-benzylthio-6-(2,4,6-trifluorophenyl)-[1,2,4]-triazolo[1,5-a]pyrimidine

A mixture of 3-benzylthio-5-amino-1,2,4-triazole (0.1 mol), diethyl (2,4,6-trifluorophenyl)malonate (0.1 mol) and tributylamine (50 ml) was heated at 180° C. for 6 hours. The reaction mixture was cooled, aqueous NaOH solution was added (21 g in 200 ml of H₂O) and the mixture was stirred for about 30 min. The phases were separated and the aqueous phase was then washed with diethyl ether and acidified with conc. HCl solution. The precipitate gave, after filtration and drying, 30 g of the starting compound of m.p. 273° C.

### Example 3

### Preparation of 5,7-dichloro-2-benzylthio-6-(2,4,6-trifluorophenyl)-[1,2,4]-triazolo[1,5-a]pyrimidine

0.05 mol of 5,7-dihydroxy-2-benzylthio-6-(2,4,6-trifluorophenyl)-[1,2,4]-triazolo[1,5-a]pyrimidine (Ex. 2) in 50 ml of phosphorus oxychloride was heated under reflux for 8 hours. During this time, some of the phosphorus oxychloride was distilled off. The residue was poured into a mixture of dichloromethane and water and boiled. The organic phase was then separated off. Drying and removal of the solvent by distillation gave 18 g of the title compound of m.p. 106° C.

### Example 4

### Preparation of 5-chloro-7-(4-methylpiperidin-1-yl)-2-benzylthio-6-(2,4,6-trifluorophenyl)-[1,2,4]-triazolo[1,5-a]pyrimidine

With stirring, a mixture of 4-methylpiperidine (10 mmol), triethylamine (10 mmol) and dichloromethane (10 ml) was added to a solution of 10 mmol of 5,7-dichloro-2-thiomethyl-6-(2,4,6-trifluorophenyl)-[1,2,4]-triazolo[1,5-a]pyrimidine (Ex. 3) in 20 ml of dichloromethane. After 16 hours of stirring at 20-25° C., the reaction solution was washed with 5% strength HCl solution. The organic phase was separated off and dried, and the solvent was removed. The residue gave, after chromatography on silica gel, 4.0 g of the title compound of m.p. 147° C.

### Example 5

### Preparation of 5-chloro-7-(4-methylpiperidin-1-yl)-2-mercapto-6-(2,4,6-trifluorophenyl)-[1,2,4]-triazolo[1,5-a]pyrimidine (method 1)

A mixture of 0.5 mmol of the triazolopyrimidine from Ex. 4 and 1.5 mmol of AlCl₃ in 15 ml of benzene was stirred at 20-25° C. for about 4 hours. The product was precipitated by addition of in each case 15 ml of methyl tert-butyl ether (MTBE) and water and then filtered off. The residue was digested in acetonitrile and then filtered off and dried. This gave 1.0 g of the title compound of m.p. 194° C.

### Example 6

### Preparation of 5-chloro-7-(4-methylpiperidin-1-yl)-2-methylsulfonyl-6-(2,4,6-trifluorophenyl)-[1,2,4]-triazolo[1,5-a]pyrimidine

A mixture of 5 mmol of 5-chloro-7-(4-methylpiperidin-1-yl)-2-thiomethyl-6-(2,4,6-trifluorophenyl)-[1,2,4]-triazolo[1,5-a]pyrimidine [cf. WO 02/088127] and 15 mmol of m-chloroperbenzoic acid (MCPA) in 20 ml of CHCl₃ was stirred at 20-25° C. for 12 hours. The solvent was distilled off and the residue was then taken up in ethyl acetate, washed with sat. NaHCO₃ solution and dried. Removal of the solvent gave 2.0 g of the title compound of m.p. 206° C.

### Example 7

### Preparation of 5-chloro-7-(4-methylpiperidin-1-yl)-2-mercapto-6-(2,4,6-trifluorophenyl)-[1,2,4]-triazolo[1,5-a]pyrimidine (method 2)

A solution of 5 mmol of the sulfone from Ex. 6 in 10 ml of dioxane was added to 10 ml of an aqueous, 20% by weight strength solution of (NH₄)₂S. After 16 hours of stirring at 20-25° C., the precipitate was filtered off and washed with water. Drying gave 1.3 g of the title compound of m.p. 194° C.

### Example 8

### Preparation of 5-chloro-7-(4-methylpiperidin-1-yl)-2-mercapto-6-(2,4,6-trifluorophenyl)-[1,2,4]-triazolo[1,5-a]pyrimidine (method 3)

A suspension of 5 mmol of the sulfone from Ex. 6 and 6 mmol of sodium thiolate hydrate in 15 ml of water was heated under reflux for one hour. After cooling, the mixture was acidified and the precipitate was then filtered off. Drying gave 1.2 g of the title compound of m.p. 194° C.

### Example 9

### Preparation of 5-chloro-7-(4-methylpiperidin-1-yl)-2-mercapto-6-(2,4,6-trifluorophenyl)-[1,2,4]-triazolo[1,5-a]pyrimidine (method 4)

A solution of 4.7 mmol of 5-chloro-7-(4-methylpiperidin-1-yl)-2-thiomethyl-6-(2,4,6-trifluorophenyl)-[1,2,4]-triazolo[1,5-a]pyrimidine [cf. WO 02/088127] and 4.7 mmol of MCPA in 20 ml of chloroform was stirred at 0° C. for about 1 hour. The solvent was distilled off and the residue was then taken up in ethyl acetate, washed with sat. NaH—CO₃ solution and dried. Removal of the solvent gave 1.7 g of the corresponding sulfoxide which was dissolved in 10 ml of trifluoroacetic anhydride. The solution was stirred at 40° C. for 1 hour and the solvent was then removed and the residue was suspended in 20 ml of a mixture of in each case 50% by volume of methanol and triethylamine. After removal of the solvent by distillation, the residue was taken up in methylene chloride, washed with sat. NH₄Cl solution and dried, and the solvent was removed. This gave 1.7 g of the title compound of m.p. 194° C.

Examples of the action against harmful fungi

The fungicidal action of the compounds of the formula I was demonstrated by the following experiments:

The active compounds were prepared as a 10% strength emulsion in a mixture of 70% by weight of cyclohexanone, 20% by weight of Nekanil®) LN (Lutensol®) AP6, wetting agent having emulsifying and dispersing action based on ethoxylated alkylphenols) and 10% by weight of Wettol® EM (nonionic emulsifier based on ethoxylated castor oil) and diluted with water to the desired concentration.

Use example: Activity against gray mold, caused by Botiytis cinerea, on bell pepper leaves, protective application

Bell pepper seedlings of the cultivar “Neusiedler Ideal Elite” were, after 4 to 5 leaves were well-developed, sprayed to runoff-point with an aqueous suspension having the concentration of active compound stated below. The next day, the treated plants were inoculated with a spore suspension of Botrytis cinerea, which contained 1.7×10⁶ spores/ml in an aqueous 2% strength biomalt solution. The test plants were then placed in a climatized chamber at 22-24° C. and high atmospheric humidity. After 5 days, the extent of the fungal infection on the leaves could be determined visually in %.

Evaluation was carried out by determining the infected leaf areas in percent. These percentages were converted into efficacies.

The efficacy (E) is calculated as follows, using Abbot's formula:
E=(1−α/β)·100
α corresponds to the fungal infection of the treated plants in % and
β corresponds to the fungal infection of the untreated (control) plants in %

At an efficacy of 0, the degree of infection of the treated plants corresponds to that of the untreated control plants; at an efficacy of 100, the treated plants were not infected.

In this test, the untreated plants were 45% infected. The preparation which contained 250 ppm of the compound from Example 9 had an efficacy of 56%.

1. A 2-mercapto-substituted triazolopyrimidine of the formula I 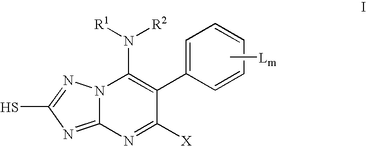
in which the substituents are as defined below:
L independently of one another are halogen, cyano, nitro, C₁-C₆-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy or —C(═O)-A;
A is hydrogen, hydroxyl, C₁-C₈-alkyl, C₂-C₈-alkenyl, C₁-C₈-alkoxy, C₁-C₆-haloalkoxy, C₁-C₈-alkylamino or di-(C₁-C₈-alkyl)amino;
m is 0, 1, 2, 3, 4 or 5;
X is halogen, cyano, C₁-C₄-alkyl, C₁-C₄-haloalkyl, C₁-C₄-alkoxy or C₁-C₂-haloalkoxy;
R¹, R² independently of one another are hydrogen, C₁-C₈-alkyl, C₁-C₈-haloalkyl, C₃-C₆-cycloalkyl, C₃-C₆-halocycloalkyl, C₂-C₈-alkenyl, C₄-C₁₀-alkadienyl, C₂-C₈-haloalkenyl, C₃-C₆-cycloalkenyl, C₂-C₈-alkynyl, C₂-C₈-haloalkynyl or C₃-C₆-cycloalkynyl, phenyl, naphthyl or a five- to ten-membered saturated, partially unsaturated or aromatic heterocycle which contains one to four hetero atoms from the group consisting of O, N and S,
R¹ and R² together with the nitrogen atom to which they are attached may also form a five- or six-membered ring which may be interrupted by one atom from the group consisting of O, N and S and/or may carry one or more substituents from the group consisting of halogen, C₁-C₆-alkyl, C₁-C₆-haloalkyl and oxy-C₁-C₃-alkyleneoxy or in which a nitrogen atom and an adjacent carbon atom may be linked by a C₁-C₄-alkylene chain;
where R¹ and/or R² may be substituted by one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₃-C₆-cycloalkyl, phenyl, naphthyl, a five- to ten-membered saturated, partially unsaturated or aromatic heterocycle which contains one to four hetero atoms from the group consisting of O, N and S,
where these aliphatic, alicyclic or aromatic groups for their part may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups in these radicals contain 2 to 8 carbon atoms;
and/or one to three of the following radicals:
cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the alkyl radicals preferably contain 6 to 10 ring members and the hetaryl radicals 5 or 6 ring members, where the cyclic systems may be partially or fully halogenated or substituted by alkyl or haloalkyl groups,
or a salt thereof.

2. A compound of the formula I as claimed in claim 1 in which X is halogen.

3. A compound of the formula I as claimed in claim 1 or 2 in which R¹ and R² are as defined below:
R¹ is C₁-C₆-alkyl, C₁-C₈-haloalkyl, C₃-C₆-cycloalkyl, C₃-C₆-halocycloalkyl, C₂-C₈-alkenyl, C₂-C₈-haloalkenyl, C₂-C₈-alkynyl; and
R² is hydrogen or C₁-C₄-alkyl; or
R¹ and R² together with the nitrogen atom to which they are attached may also form a five- or six-membered saturated or unsaturated ring which may carry one or two substituents from the group consisting of halogen, C₁-C₆-alkyl and C₁-C₆-haloalkyl.

4. A compound of the formula I as claimed in claim 1 in which the phenyl group substituted by Lₘ is the group A 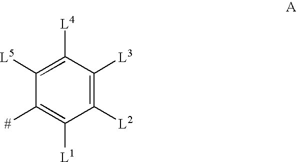
in which # is the point of attachment to the triazolopyrimidine skeleton and
L¹ is fluorine, chlorine, CH₃ or CF₃;
L², L⁴ independently of one another are hydrogen or fluorine;
L³ is hydrogen, fluorine, chlorine, cyano, CH₃ or COOCH₃; and
L⁵ is hydrogen, fluorine or CH₃.

5. A process for preparing the compounds of the formula I as claimed in claim 1 by reacting sulfoxides of the formula II 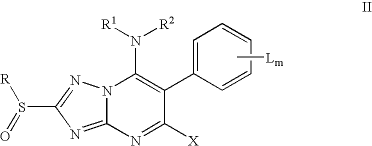
in which the variables are as defined for formula I and R is a C₁-C₄-alkyl group or a benzyl group which is unsubstituted or substituted by one or more groups R⁶ with trifluoroacetic anhydride.

6. A process for preparing the compounds of the formula I as claimed in claim 1 by reacting sulfones of the formula III 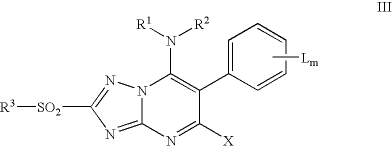
in which the variables are as defined in formula I with alkali metal thiolates or with sulfides M₂S, where M is a cation from the group of the alkali metals or an ammonium group.

7. A process for preparing the compounds of formula I as claimed in claim 1 by reacting triazolopyrimidines of the formula IV 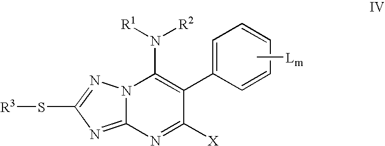
in which R³ is a benzyl group which is unsubstituted or substituted by one or more groups R^{b}
with Lewis acids or under basic conditions in an inert solvent or diluent.

8. A process for preparing the compounds of the formula I as claimed in claim 1 by reacting triazolopyrimidines of the formula IV as set forth in claim 7 with sodium in liquid ammonia.

9. A composition suitable for controlling harmful fungi which composition comprises a solid or liquid carrier and a compound of the formula I as claimed in claim 1.

10. A method for controlling phytopathogenic harmful fungi which comprises treating the fungi or the materials, plants, the soil or seeds to be protected against fungal attack with an effective amount of a compound of the formula I as claimed in claim 1.
